(19) United States
(12) Patent Application Publication
(10) Pub. No.: US 2013/0346007 A1
(43) Pub. Date: Dec. 26, 2013

(54) Characterizing an Elongated Textile Test Material

(71) Applicant: Uster Technologies, AG, Uster (CH)

(72) Inventors: Peter Schmid, Zurich (CH); Beat Keller, Zurich (CH); Stefan Gehrig, Uster (CH); Rafael Storz, Kreuzlingen (CH); Flavio Carraro, Saland (CH)

(21) Appl. No.: 14/004,034

(22) PCT Filed: Mar. 14, 2012
(86) PCT No.: PCT/CH12/00059

§ 371 Date: Sep. 9, 2013

(30) Foreign Application Priority Data
Mar. 6, 2011 (CH) 460/11

(51) Int. Cl. G01N 33/36; G06F 17/21

(52) U.S. Cl. CPC G01N 33/365; G06F 17/21
USPC 702/84; 715/273

(57) ABSTRACT

The measured values of a property of a yarn along its longitudinal direction are detected for the characterization of a yarn moved along its longitudinal direction. The values of a parameter of the yarn are determined from the measured values. An event field is provided, whose abscissa indicates an extension of parameter values in the longitudinal direction and whose ordinate indicates a deviation of the parameter from a set point value. Densities of events in the event field are determined from the values of the parameter and its extension in the longitudinal direction. A test material body is calculated in the event field as an area, which is delimited by the abscissa on the one hand, by the ordinate on the other hand, and further by a line which substantially follows a constant event density. The area is specified numerically. At least one value of the numerical specification is output as a characteristic of the yarn.

### BACKGROUND

The present invention lies in the field of textile quality control. It relates to a method and an apparatus for characterizing an elongated textile test material, according to the preambles of the independent claims. Such methods and apparatuses are typically used in spinning or winding machines. The elongated textile test material is preferably a yarn, but can also be a sliver or a roving, etc.

The invention also relates to a quality reference document, containing quality data for an elongated textile test material.

So-called yarn clearers are used in spinning or winding machines for securing the yarn quality. Such an apparatus is known for example from EP-0′439′767 A2. It contains a measuring head with at least one sensor which scans the moved yarn. Frequently used sensor principles are the capacitive one (see EP-0′924′513 A1 for example) or the optical one (see WO-2004/044579 A1 for example). It is the object of scanning to detect defects such as thick places, thin places or foreign substances in the yarn. The output signal of the sensor is evaluated continuously with predetermined evaluation criteria. The evaluation criteria are usually predetermined in form of a clearing limit or clearing curve in a two-dimensional event field which is spanned by the length of the event on the one hand and by an amplitude of the event (e.g. the deviation of the yarn mass from a set point value) on the other hand. Events beneath the clearing limit are tolerated, whereas events above the clearing limit are removed from the yarn or at least recorded as defects.

It has been common practice for decades to classify yarn defects with the USTER® CLASSIMAT system of the applicant of the present protective right. This system is described for example in the U.S. Pat. No. 5,537,811 A and in the brochure “USTER® CLASSIMAT QUANTUM”, Uster Technologies AG, August 2007. Accordingly, the aforementioned event field is subdivided into a discrete number (e.g. 23) of rectangular classes, thereby producing a classification field. Each yarn defect can then be associated to a class according to its length and amplitude. The determined yarn defects in each class are counted and converted into a standard yarn length of 100 km for example. The totality of the numerical values thus obtained in each class characterizes the yarn and can then be used for determining a clearing threshold. This classification supplies a relatively high number of starting values on the one hand (namely the 23 numerical values for example), but it is nevertheless relatively rough on the other hand because the events are no longer distinguished further within the individual classes.

WO-2010/078665 A1 describes a method and an apparatus for characterizing a textile test material moved along its longitudinal direction. In this process, the measured values of one property of the test material are detected along its longitudinal direction. The values of a test material parameter are determined from the measured values. The densities of events in the event field are determined from the values of the test material parameter and its extension in the longitudinal direction. A test material body is graphically displayed in the event field as an area. The area is bounded by the abscissa on the one hand, by the ordinate on the other hand, and further by a line in the event field which substantially follows a constant event density. The illustration of the test material body allows an operator to rapidly determine the characteristic properties of the test material and to rationally predetermine a clearing limit.

### SUMMARY

It is an object of the present invention to further develop the method and the apparatus according to WO-2010/078665 A1 in such a way that the characterization of the textile test material will improve and be more objective.

These and other objects are achieved by the method and the apparatus in accordance with the invention as defined in the independent claims. Advantageous embodiments are provided in the dependent claims.

The invention utilizes the concept of the test material body introduced in WO-2010/078665 A1, which can be displayed in the two-dimensional event field as an area bounded by a density curve. It is the basic idea of the invention to make this graphic structure numerically comprehensible in a suitable way. This can occur for example by means of supporting points, a curve fitting or in any other way.

Accordingly, in the inventive method for characterizing an elongated textile test material moved along its longitudinal direction measured values of a property of the textile test material are determined along the longitudinal direction of the textile test material. Values of a parameter of the textile test material are determined from the measured values. An event field is provided which contains a quadrant or a part of a quadrant of a two-dimensional Cartesian coordinate system, whose abscissa indicates an extension of parameter values in the longitudinal direction and whose ordinate indicates a deviation of the parameter from a set point value. Densities of events in the event field are determined from the values of the parameter and their extension in the longitudinal direction. A test material body is calculated as an area in the event field, which area is bounded by the abscissa or a straight line extending parallel thereto on the one hand, by the ordinate or a straight line extending parallel thereto on the other hand, and further by a line in the event field which substantially follows a constant event density. The area is specified numerically. At least one value of the numeric specification is output as a characteristic of the textile test material.

The term of “area” shall be understood in this specification as a subset of the plane which is spanned by the two-dimensional Cartesian coordinate system. The area defined in this manner shall be distinguished from the area content, which is a measure for the size of the area and therefore a property of the area. The area representing the test material body is preferably connected. The term of “connected” can certainly be understood in this case within the terms of the mathematical topology. It should not play any role in practice whether the general connectedness or the more special path connectedness is used for the definition. However, the area does not need to be simply connected, i.e., it may also comprise recessed portions which are enclosed on all sides.

The numerical specification can consider the following properties of the area for example:
area content of the area,
geometrical shape of the area, e.g. position of center of area or center of contours of the area, and/or
a progression of the line which further limits the area. The numerical specification can occur by means of supporting points. A specific supporting-point-based specification system is defined by predetermining the number of the used supporting points and their position on the abscissa.

If the mentioned line is considered, the numerical specification can occur by means of an adjustment calculus. Respective mathematical methods of adjustment calculus are known. Preferably, data points lying on the line are chosen and approximated by a fit function by taking the method of the smallest squares into account. A specific fit-based specification system is defined by predetermining the fit function. Various fit functions can be considered, whose common feature should be the decline in large abscissa values. The resulting function parameter values are then used for characterizing the examined test material.

For the purpose of specification by means of a line, irrespective of whether it is the mentioned line or a fit curve, at least one turning point, at least one inflection point, line centroid and/or at least one gradient can be calculated. Further numerical values as known from mathematical curve sketching can be indicated.

It may be advantageous for the numerical specification to output a confidence region, preferably in form of confidence intervals or a confidence area. The constant event density, which corresponds to the mentioned line, should fulfill the following criteria:
It is so high that the removal of all events from the test material which occur with this or a lower event density would impair productivity too much, and
it is so low at the same time that it still lies sufficiently close to the fault density the removal of which from the test material appears to make sense, e.g. between 10 and 100 times higher.

The event density will preferably be related to a Cartesian coordinate system with axes divided in a double logarithmic manner. U.S. Pat. No. 6,374,152 B1 deals in detail with event densities. Threshold event densities, which are especially suitable for yarn, lie between 100 and 3,000 events per 100 km of test material length, and preferably at 1000 events per 100 km of test material length.

The parameter preferably substantially corresponds to a mass per unit of length or a diameter of the textile test material. Alternatively, it can relate to a reflectivity or an absorptivity of the textile test material, foreign substances in the test material, or any other property of the textile test material.

Several different areas (e.g. two), which are preferably situated in different quadrants of the event field, can be specified numerically in the event field. The several areas can individually be specified numerically. Alternatively, the several areas can be combined into one single area and the single area produced by the combination can be specified numerically.

The at least one value of the numerical specification can be included in a quality reference document for the respective test material, such as USTER® STATISTICS of the applicant of the present protective right. For this purpose, a set of samples of the respective test material of the same type, which set is representative for worldwide production, is preferably collected and characterized according to the present invention. Percentiles, relating to the worldwide production of the respective test material, can be indicated for example in the quality reference document for the at least one value of the numerical specification, preferably in form of a nomogram. A quality reference document in accordance with the invention with quality data for an elongated textile test material accordingly contains a characterization of the textile test material which was obtained according to the method in accordance with the invention as described above. As a result, the invention allows classifying the quality of the textile test material in an even more comprehensive manner with respect to a representative set of test materials of the same type which were produced at another location and/or at another time.

The apparatus in accordance with the invention is used for characterizing an elongated textile test material moved along its longitudinal direction. It contains a measuring head for detecting measured values of a property of the textile test material along the longitudinal direction of the textile test material and for determining values of a parameter of the textile test material from the measured values. Furthermore, it contains a control unit which is connected to the measuring head. The control unit has a memory unit and an output unit for storing and outputting an event field which contains a quadrant or a part of a quadrant of a two-dimensional Cartesian coordinate system, whose abscissa indicates an extension of parameter values in the longitudinal direction and whose ordinate indicates a deviation of the parameter from a set point value. Moreover, the control unit comprises a computing unit which is configured to determine densities of events in the event field from the values of the parameter and their extension in the longitudinal direction. The computing unit is also configured to calculate a test material body as an area in the event field, which area is bounded on the one hand by the abscissa or a straight line extending parallel thereto, and on the other hand by the ordinate or a straight line extending parallel thereto, and further by a line in the event field which substantially follows a constant event density. The control unit is configured to numerically specify the area and to output at least one value of the numerical specification as a characteristic of the textile test material.

The apparatus in accordance with the invention can be used in a textile processing machine, e.g. a spinning or winding machine for yarn. Such a textile processing machine typically comprises a plurality of working stations. Accordingly, the apparatus in accordance with the invention may contain a plurality of measuring heads which are located at each working station. The measuring heads are all connected to the central control unit, e.g. via a serial bus such as RS-485. An interface converter can be installed between a respective measuring head and the control unit. The control unit is preferably installed in the textile processing machine.

Whereas the publication WO-2010/078665 A1, which is known from the state of the art, allows intuitive detection of the test material characteristics by graphical display of the test material body, the present invention characterizes the test material precisely by numerical values. This is performed however in an entirely different and simpler way than with the system USTER® CLASSIMAT, which is also known from the state of the art. The latter system requires numerical values in 23 or more classes for characterization. The present invention however makes do with very few (e.g. 2 to 6) parameters and therefore reduces the data quantity required for the characterization. Furthermore, the characterization in accordance with the invention is even more precise under certain circumstances than the one of USTER® CLASSIMAT, because it supplies values from a continuous set of values, rather than numerical values relating to discrete classes.

### DRAWINGS

The invention will be explained below in closer detail by reference to an example of a winding machine for yarn shown in the drawings. This example shall not limit the generality because the invention can also be applied similarly well to other elongated textile test materials such as slivers or rovings.

FIG. 1 schematically shows a winding machine with a yarn clearer system.

FIGS. 2-7 each show an event field with one or two yarn bodies and the numerical specification according to the method in accordance with the invention.

FIG. 8 shows nomograms with percentiles for quality classification of a yarn characterized in accordance with the invention with respect worldwide yarn production.

### DESCRIPTION

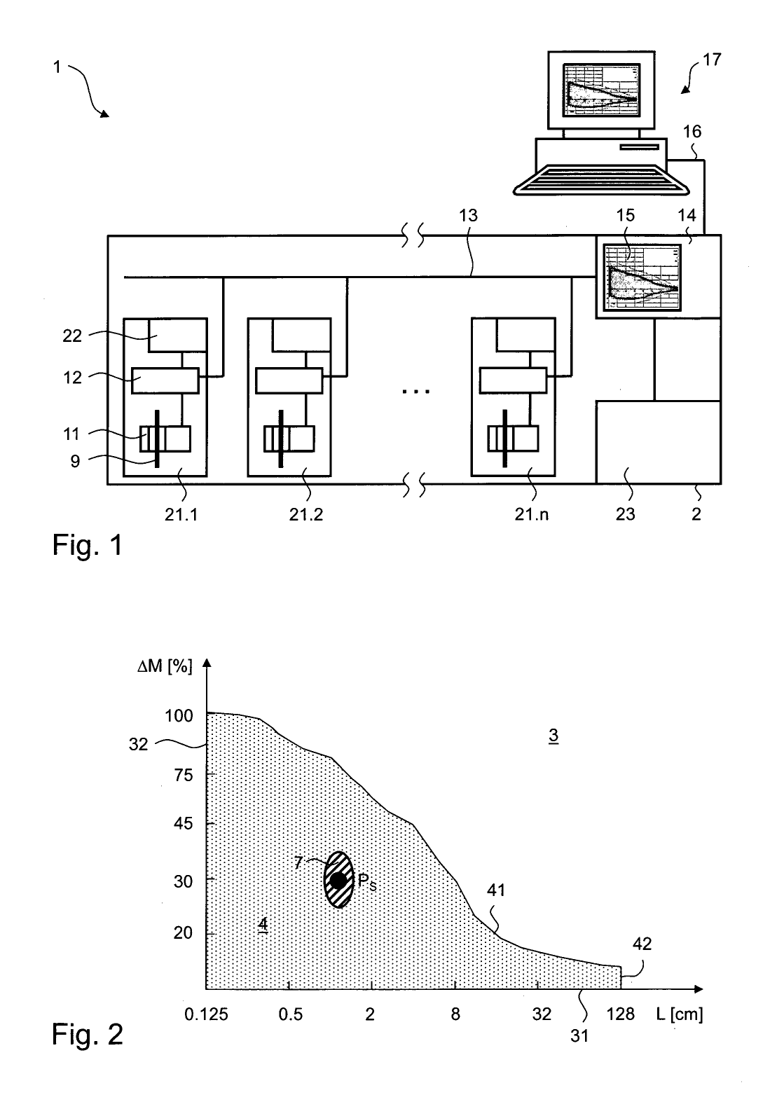

FIG. 1 shows a highly schematic view of a winding machine 2 with several winding stations 21.1, 21.2, . . . 21.n. An apparatus 1 in accordance with the invention is installed in the winding machine 2. Yarn 9 is monitored by a measuring head 11 of the apparatus 1 in accordance with the invention at each winding station 21.1 during the rewinding process. The measuring head 11 contains a sensor with which a property of the yarn 9 is measured, e.g. a capacitive sensor for measuring a dielectric property of the yarn 9. Furthermore, the measuring head 11 contains an evaluation unit which is configured to determine a yarn parameter such as the yarn mass per unit of length from the measured values. The measuring head 11 is connected via an interface converter 12 to a central control unit 14 of the apparatus 1 in accordance with the invention. The measuring head 11 is set and controlled via the connection by the control unit 14, and the measuring head 11 transmits data such as the determined yarn parameters to the control unit 14. A connecting line 13 between all interface converters 12 and the control unit 14 can be arranged as a serial bus such as RS-485. The interface converter 12 can additionally also be connected to a winding station computer 22 of the respective winding station 21.1. The control unit 14 comprises an output unit and an input unit for an operator. Preferably, the output and the input unit are jointly arranged as a sensor screen (touchscreen) 15. The control unit 14 is connected to a control computer 23 of the winding machine 2. The output and/or the input unit can be installed in the winding machine 2, e.g. in the control computer 23, instead of in the control unit 14.

The control unit 14 is connected via a data line 16 to a computer station 17. The computer station 17 is independent and preferably configured as a personal computer (PC) with input and output units. It is not mandatory for the invention, but advantageous in order to perform at least in part of the evaluations in accordance with the invention and/or to store the results. It can preferably exchange data, especially the numerical specification of the yarn 9, via a data network and/or via mobile data carriers with other computer stations. Alternatively, the aforementioned tasks of the computer station 17 could be assumed by the control unit 14.

FIG. 2 shows a possible event field 3 with a yarn body, as can be calculated in the control unit 14 or the computer station 17 and as can be shown on the output unit 15. The event field 3 is a quadrant of a two-dimensional Cartesian coordinate system which is spanned by an abscissa 31 and an ordinate 32. The ordinate 32 indicates a deviation ΔM of the yarn parameter, e.g. the deviation of the yarn mass per unit of length in percent, from a set point value. The set point value is preferably determined by continuous averaging over a plurality of measurements. The abscissa 31 indicates the length L along which the respective deviation ΔM extends in the longitudinal direction of the yarn. The division of the two axes 31, 32 is preferably logarithmic. A determined deviation ΔM and its length L jointly form the coordinates of a yarn event which defines a point in the event field 3 and can be represented there in a suitable manner.

A sufficiently long yarn section is measured in a calibration process. As “Sufficient” shall be regarded a calibration length of at least approximately 1 km, larger calibration lengths of 10 km or 100 km for example are preferred because they supply statistically more meaningful results. The values of the yarn parameter and the associated lengths L are transferred by the measuring head 11 to the control unit 14. The densities of events in the event field 3 are determined therefrom in a computing unit of the control unit 14, as described for example in U.S. Pat. No. 6,374,152 B1. The event densities preferably relate to a Cartesian coordinate system with axes that are divided in a double logarithmic manner, as shown in FIGS. 2 to 7. As a result, each point of the event field 3 can be assigned an event density in an unequivocal manner. Excessively abrupt local changes in the event density function determined in this manner, which are possibly caused by measuring errors or other artifacts, can be prevented by interpolation, extrapolation, smoothing and/or other numeric methods.

A yarn body is calculated from the event density function and represented as an area 4 in the event field 3. For this purpose, a sufficiently high threshold event density of 1000 events per 100 km of yarn length is chosen for example. The connection of all points in the event field 3, to which the threshold event density is associated, leads to a density curve 41 which bounds the yarn body from the remaining event field 3. The yarn body is bounded by the coordinate axes 31, 32 themselves towards the two coordinate axes 31, 32. Towards large lengths L, the yarn body can also be bounded by a further line 42 which extends for example at L=128 cm parallel to the ordinate 32. As a result of these boundaries, a connected area 4 is obtained which is characteristic for the measured yarn 9. The area 4 which represents the yarn body differs graphically from the remaining event field 3, in that it has a different color, a different gray shade and/or a different pattern than the remaining event field 3. Such a calculation and representation of the yarn body are described in detail in WO-2010/078665 A1. They allow the operator to recognize the characteristics of the examined yarn in a rapid and intuitive way.

Furthermore, the present invention captures the characteristics of the examined yarn 9 in a numerical manner. For this purpose, the area 4 is specified numerically. In the embodiment of FIG. 2, the numeric specification occurs by means of a centroid P_{S} of the area 4. The coordinates p_{S} of the centroid P_{S} of a flat area 4 are calculated according to the following known formula:

$${\underline{p}}_{s} = \frac{1}{A} {\int }_{4} \underline{x} d A , \qquad ( 1 )$$

wherein integration is performed over the entire area 4, and

$$A = {\int }_{4} d A \qquad ( 2 )$$

is the area content of area 4. In the example of FIG. 2, the centroid P_{S} has the coordinates p_{S}=(1.1 cm, 29%). Furthermore, a confidence interval can be stated for each of the two coordinate values, which is determined by one of the common statistical methods, e.g. δ(L)=±0.3 cm and δ(ΔM)=±5%. In FIG. 2, an elliptical confidence area 7 is shown around the centroid P_{S}, whose projections on the respective axis correspond to the above confidence intervals. The numerical specification of the area 4 of FIG. 2 is therefore: p_{S}=((1.1±0.3) cm, (29±5) %).

The area 4 can be specified numerically with the position of its contour centroid instead of the area centroid P_{S} or in addition to the same. The contour centroid can be defined with integrals in analogy to Formulas (1) and (2), wherein integration is performed over the contour lines which bound the area 4 instead of over the area 4.

The area 4 can be specified alternatively or additionally by its area content A according to the Formula (2), wherein a confidence interval can also be stated for the area content A.

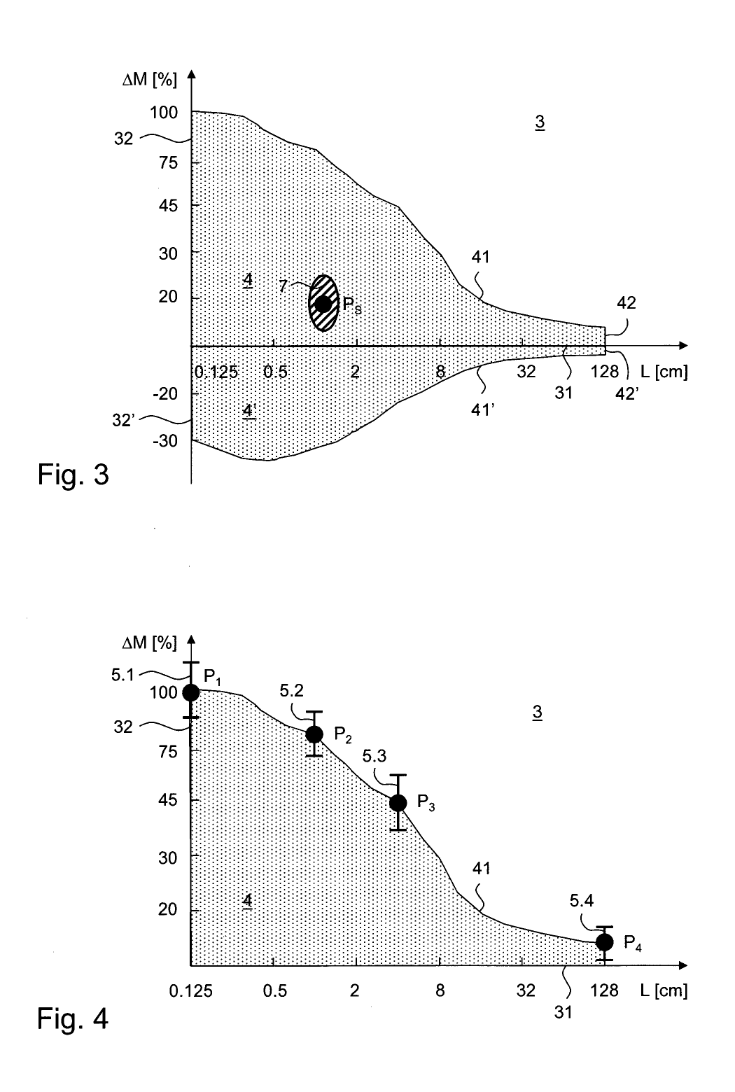

FIG. 3 shows a second quadrant of the event field 3 in addition to the first quadrant, whose abscissa 31 is identical with that of the first quadrant, but whose ordinate 32′ extends towards negative values of the deviation ΔM. Accordingly, the events in the first quadrant are thick places in the yarn 9, and thin places in the second quadrant. An area 4, 4′ which represents the respective yarn body can be calculated in each of the two quadrants. The second area 4′ in the second quadrant can be specified numerically in the same manner as the first area 4 in the first quadrant, e.g. with the area centroid, the contour centroid or the area content. Alternatively, the two areas 4, 4′ can be combined into one single area, and the single area resulting from the combination is numerically specified. An area centroid P_{S} for the combined area is shown in FIG. 3, together with a confidence area 7. The centroid coordinates are p_{S}=(1.1 cm, 19%) in this example. In any case, the two areas 4, 4′ characterize the examined yarn even more completely than the first area 4 for thick places alone.

In the embodiment of FIG. 4, the area 4 is numerically specified by means of the density curve 41, and further by the supporting points P₁-P₄. A specific supporting-point-based specification system is defined by predetermining the number of the used supporting points and their positions on the abscissa 31. The example of FIG. 4 comprises four supporting points P₁-P₄ at the lengths L₁=0.125 cm, L₂=1 cm, L₃=4 cm and L₄=128 cm. The y values of these supporting points P₁-P₄ are in this example ΔM₁=100%, ΔM₂=80%, ΔM₃=45% and ΔM₄=8%. They lie on the density curve 41 and therefore substantially have the same threshold event density. It is advantageous to additionally provide a confidence interval δ(ΔM) for each supporting point P₁-P₄. The confidence intervals δ(ΔM) can be determined with one of the common statistical methods and are shown in FIG. 4 in form of error bars 5.1-5.4. The yarn body or the area 4 of FIG. 4 is therefore specified by the following eight quantities:

| Supporting | | |
|---|---|---|
| point | ΔM [%] | δ(ΔM) [%] |
| P₁ | 100 | ±16 |
| P₂ | 80 | ±12 |
| P₃ | 45 | ±16 |
| P₄ | 8 | ±8 |

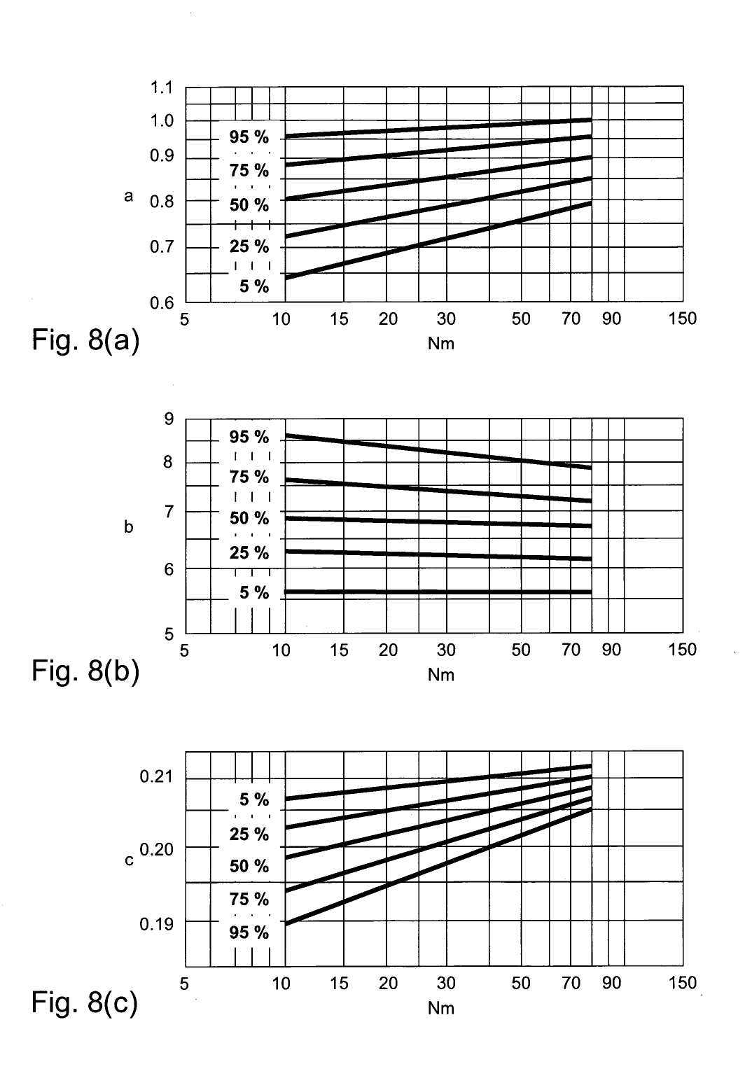

It is understood that more or less than four supporting points can be used. The position of the supporting points will be chosen in such a way that the fewest number of supporting points characterize the yarn 9 in the best possible way. Certain areas on the abscissa 31 can be uninteresting, e.g., because only few yarn defects occur in them or because the respective yarn defects are perceived to offer little disturbance. The question as to whether and to which areas this applies depends on the respective type of yarn, the intended use of the yarn and possibly on further factors. In any case, the person skilled in the art will be able, with the knowledge of the present invention, to indicate in a given situation as few as possible supporting points that are characteristic to the highest possible extent. It is possible that the person skilled in the art will come to an agreement on a specific supporting-point-based specification system and will use it in order to exchange quality data of yarns among each other. The ΔM values which belong to the thus predetermined supporting points could then also be included in a quality reference document and be displayed in nomograms in the manner of FIG. 8.

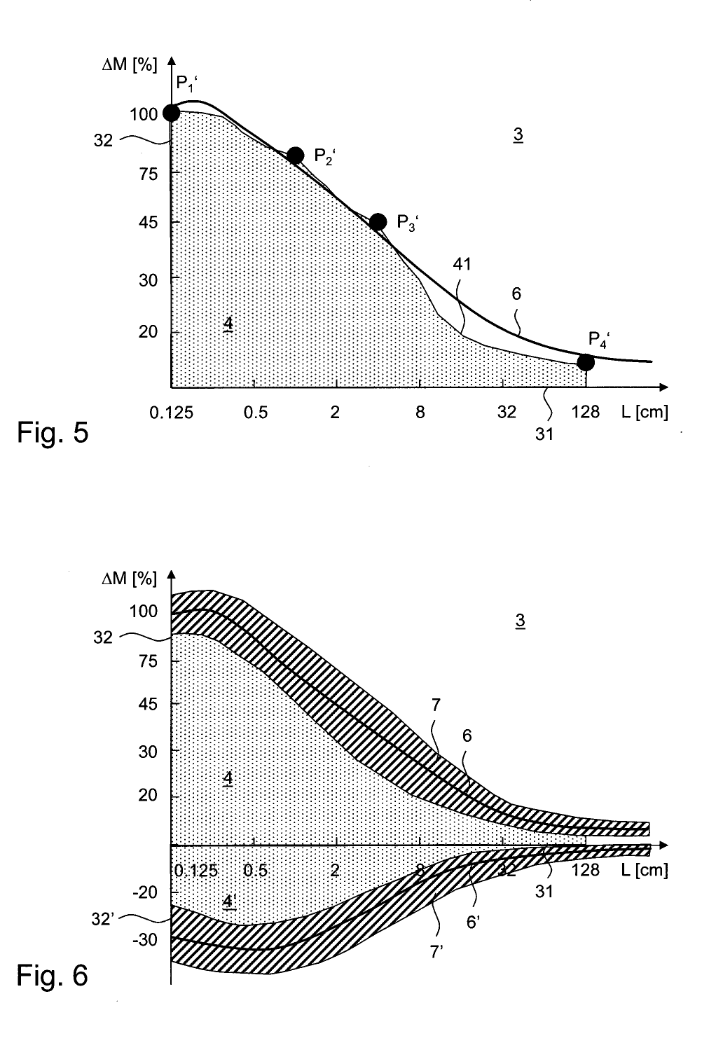

A different kind of the numerical specification of the area 4 on the basis of the density curve 41 is shown in FIG. 5. In this case, the density curve 41 is adjusted by a fit curve 6. The adjustment can occur by a known method of adjustment calculus. Preferably, the characteristic data points P₁′-P₄′ are chosen on the density curve 41, and the data points P₁′-P₄′ are approximated with a function fit by taking the method of the smallest squares into account. A specific fit-based specification system is defined by predetermining the fit function. Two suitable fit functions are presented below by way of example, whose common aspect is that they have three parameters and decrease for large L values.

A first example for a suitable fit function is the function

y=a(x+b)e^{-cx}, (3)

wherein x=ld(L) (logarithm of L to the base 2), y=ld(|ΔM|), and a, b as well as c are the function parameters to be found. The yarn body of FIG. 3 can be specified by the following parameter values for example:

a=0.82,

b=7.6,

c=0.20.

It is possible that not all function parameters a, b, c of the respective fit function (3) are of the same relevance for the characterization of the yarn 9. Function parameters which have proven in practice to be insignificant do not need to be output, thus advantageously reducing the number of parameter values which are needed for the characterization of the yarn 9. As a result, a fit function could be used with four function parameters, of which two hardly correlate with the yarn quality, so that only two function parameters characterize the yarn 9, but still better than the three function parameters a, b, c of the above fit function (3).

A second example for a fit function is the bell-shaped curve

y=f·e^{-g(x-h)}^{²}, (4)

wherein again x=ld(L), y=ld(|ΔM|), and f, g as well as h are the function parameters to be found. The following can be given as exemplary parameter values:

f=6.7,

g=0.010,

h=−2.5.

It is also advantageous in the fit-based specification to provide a confidence area. Such a confidence area is shown in FIG. 6 as a hatched area 7. The confidence area 7 can be a band for example in which the fit curve 6 is situated. The width of the confidence band 7 can be chosen in such a way that the “true” density curve lies with a certain probability of 95% for example within the confidence band 7. The confidence band 7 can again be specified with numerical values, e.g., by indicating confidence intervals for the resulting function parameters, by indicating the functions ΔM(L) for its upper and a bottom boundary line or by indicating the distance of the boundary lines from the fit curve 6.

Similar to FIG. 3, a second quadrant of the event field 3 is also shown in FIG. 6 in addition to the first quadrant, in which a fit curve 6′ for thin places and a respective confidence band 7′ is placed. If a supporting-point-based specification system is used (see FIG. 4), the same or different supporting points can be used in both quadrants. In the case of a fit-based specification system (see FIG. 5), the same fit function or different fit functions can be used for both quadrants. The yarn body of FIG. 6 is specified with the following six (or twelve—if the confidence intervals are stated) quantities by using the above fit function (3):

| Parameter | Thick places | Thin places |
|---|---|---|
| a | 0.82 ± 0.12 | −0.99 ± 0.12 |
| b | 7.6 ± 1.5 | 5.5 ± 0.9 |
| c | 0.20 ± 0.02 | 0.20 ± 0.03 |

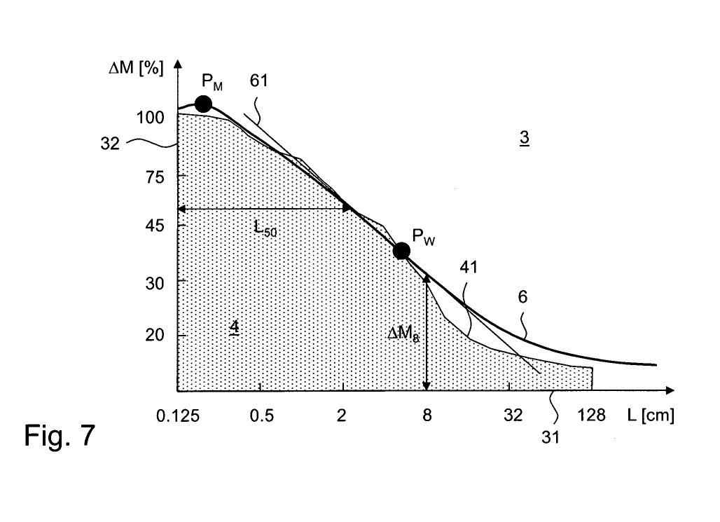

FIG. 7 shows further possibilities of the numerical specification of the area 4 on the basis of the density curve 41. It is assumed that the density line 41 (as already in FIG. 5) was adjusted by a fit curve 6. Further numerical values of the fit curve 6 are calculated here instead of the function parameters a to c of the fit function (3), or in addition to the same, and are output as a characteristic of the yarn (9). Such numerical values can be function values and/or values of the derivatives, as considered in mathematical curve sketching. Examples are as follows:
Turning points. The fit curve 6 has a maximum in point P_{M} with the coordinates p_{M}=(0.18 cm, 119%).
Inflection points. The fit curve 6 has an inflection point in point P_{W} with the coordinates p_{W}=(5.3 cm, 34%).
A line centroid of a part of the fit curve 6, e.g. the part between L=0.125 cm and L=128 cm.
Gradients of the fit curve 6. The gradients can be calculated at a fixed length L, a fixed height ΔM and/or at a variable point such as the inflection point P_{W}. FIG. 7 shows a tangent 61 by way of example with negative gradient in the inflection point P_{W}.
Points in which the fit curve 6 exceeds or falls beneath a specific value.
Point in which the gradient of the fit curve 6 exceeds or falls beneath a specific value.
An L value for a fixed height of ΔM=50% for example. FIG. 7 shows the value of L₅₀=2.3 cm by way of example. The L value can be calculated alternatively or additionally for other ΔMs which are fixed or variable. One example for the latter case is the L value at half the maximum height.
A ΔM value for a fixed length of L=8 cm for example. FIG. 7 shows the value of ΔM₈=27% by way of example. The ΔM value can be calculated alternatively or additionally for other lengths which are fixed or variable. One example for the latter case is the ΔM value at half the turning point length.

Various possibilities for the numerical specification of the yarn body can be combined with one another and thus be applied simultaneously. For example, the abscissa 31 can be divided into three areas. A first fit function for the density curve 41 can be used in a first area, supporting points can be used in a second area, and a second fit function which differs from the first fit function can be used in a third area. The possibilities for specification by means of the density line 41 as discussed above can also be applied in the specification by means of the fit curve 6, and vice versa.

In the same manner as the thick places and thin places, further quantities measured on the yarn 9 can be considered if necessary, e.g. a foreign substance signal.

The numerical specification of the yarn body can be included in a yarn quality reference document such as USTER® STATISTICS. For this purpose, a set of yarn samples of yarn of the same type which is representative for the worldwide yarn production is collected for this purpose at first and is characterized according to the present invention. The numerical results of the characterization are noted in nomograms, as shown in FIG. 8. This embodiment relates to the above fit function (3) with the three function parameters a, b, c. The FIG. 8 (a)-(c) show nomograms in which one of the parameters a, b, c is entered in relation to the metric yarn count Nm (length in kilometers per kilogram of yarn). The lines in the nomograms indicate the 5, 25, 50, 75 and 95 percentiles, relating to the worldwide yarn production, as a function of the yarn count Nm. This means: 5% of all yarns produced worldwide with a specific yarn count have parameter values beneath (or above in the example of FIG. 8 (c)) the 5 percentile, 25% of all yarns produced worldwide have parameter values beneath (or above in the example of FIG. 8 (c)) the 25 percentile, etc. The 50 percentile indicates the worldwide median. Nomograms such as shown in FIG. 8 by way of example allow quality classification of a yarn 9 characterized in accordance with the invention with respect to worldwide yarn production.

As already mentioned above, less meaningful function parameters need not be output separately. It has also been mentioned above that instead of function parameters a, b, c it is possible to display the ordinate values ΔM₁, ΔM₂, . . . of supporting points P₁, P₂, . . . in such nomograms. The output of the numerical specification can occur in a different way instead of in nomograms, either in another graphical type display or in form of numerical values which are summarized in a table for example.

It is understood that the present invention is not limited to the embodiments as discussed above. The person skilled in the art will be able to derive further variants with knowledge of the invention, which shall also belong to the subject matter of the present invention.

### REFERENCES

1 Apparatus
11 Measuring head
12 Interface converter
13 Connecting line
14 Control unit
15 Sensor screen as an input and output unit
16 Data line
17 Computing station
2 Winding machine
21.1, 21.2, . . . Winding stations
22 Winding station computer
23 Control computer
3 Event field
31 Abscissa
32 Ordinate
4 Yarn body, the area representing the yarn body
41 The density curve delimiting the event field
42 Further line delimiting the event field
5.1, 5.2, . . . Error bars
6 Fit curve
61 Tangent
7 Confidence area
9 Yarn

1. A method for characterizing an elongated textile test material that is moved along its longitudinal direction, the method comprising the steps of:
detecting measured values of a property of the textile test material along the longitudinal direction of the textile test material,
determining values of a parameter of the textile test material from the measured values,
creating an event field in a two-dimensional Cartesian coordinate system, whose abscissa indicates an extension of parameter values in the longitudinal direction and whose ordinate indicates a deviation of the parameter from a set point value,
determining densities of events in the event field from the values of the parameter and its extension in the longitudinal direction, and
calculating a test material body as an area in the event field, which area is bounded,
by at least one of the abscissa and a straight line extending parallel thereto,
by at least one of the ordinate and a straight line extending parallel thereto, and
by a line in the event field that substantially follows a constant event density,
wherein,
the area is specified numerically, and
at least one value of the numerical specification is output as a characteristic of the textile test material.

2. The method according to claim 1, wherein the numerical specification considers an area content of the area.

3. The method according to claim 1, wherein the numerical specification considers a geometrical shape of the area.

4. The method according to claim 3, wherein the numerical specification considers at least one of a position of an area centroid of the area and a contour centroid of the area.

5. The method according to claim 1, wherein the numerical specification considers a progression of the straight line.

6. The method according to claim 5, wherein the numerical specification occurs on the basis of supporting points.

7. The method according to claim 5, wherein the numerical specification occurs on the basis of an adjustment calculus.

8. The method according to claim 5, wherein at least one turning point, at least one inflection point, and at least one of a line centroid and at least one gradient is calculated.

9. The method according to claim 1, wherein a confidence range in form of at least one of confidence intervals and a confidence area is output for the numerical specification.

10. The method according to claim 1, wherein the constant event density lies between about 100 and about 3000 events per 100 kilometers of test material length.

11. The method according to claim 1, wherein the parameter substantially corresponds to at least one of a mass per unit of length of the textile test material and a diameter of the textile test material.

12. The method according to claim 1, wherein more than one different areas that lie in different quadrants of the event field are specified numerically.

13. The method according to claim 12, wherein the more than one different areas are combined into one single area, and the single area produced by combination is specified numerically.

14. The method according to claim 1, wherein the at least one value of the numerical specification is included in a quality reference document for the respective test material.

15. The method according to claim 14, wherein a set of samples of the respective test material of the same type, which is representative for worldwide production, is collected and characterized.

16. The method according to claim 15, wherein percentiles relating to the worldwide production of the respective test material are indicated in the quality reference document for the at least one value of the numerical specification, in form of a nomogram.

17. A quality reference document, containing quality data for an elongated textile test material, the document comprising a characterization of the textile test material obtained according to the method of claim 1.

18. The quality reference document according to claim 17, wherein percentiles relating to the worldwide production of the respective test material are indicated for the at least one value of the numerical specification, in form of a nomogram.

19. An apparatus for characterizing an elongated textile material moved along its longitudinal direction, the apparatus comprising:
a measuring head for detecting measured values of a property of the textile test material along the longitudinal direction of the textile test material and for determining values of a parameter of the textile test material from the measured values, and
a control unit connected to the measuring head, comprising
a memory unit and an output unit for storing or outputting an event field that contains at least a part of a quadrant of a two-dimensional Cartesian coordinate system, whose abscissa indicates an extension of parameter values in the longitudinal direction and whose ordinate indicates a deviation of the parameter from a set point value, and
a computing unit that is configured,
to determine densities of events in the event field from the values of the parameter and its extension in the longitudinal direction, and
to calculate a test material body as an area in the event field, which area is bounded,
by at least one of the abscissa and a line extending parallel thereto,
by at least one of the ordinate or a line extending parallel thereto, and
by a line in the event field that substantially follows a constant event density,
wherein the control unit is configured,
to specify the area numerically, and
to output at least one value of the numerical specification as a characteristic of the textile test material.

20. The apparatus according to claim 19, wherein the control unit is installed in a textile processing machine.
